(19) United States
(12) Patent Application Publication
(10) Pub. No.: US 2013/0160772 A1
(43) Pub. Date: Jun. 27, 2013

(54) MULTI-FUNCTIONAL NASAL CANNULA

(71) Applicant: FARROKH TABRIZCHI, Baltimore, MD (US)

(72) Inventor: FARROKH TABRIZCHI, Baltimore, MD (US)

(21) Appl. No.: 13/475,697

(22) Filed: May 18, 2012

Related U.S. Application Data
(60) Provisional application No. 61/580,617, filed on Dec. 27, 2011.

(51) Int. Cl. A61M 15/08

(52) U.S. Cl. 128/207.18

(57) ABSTRACT

The multi-function nasal cannula includes an elongate nasalgastric tube having at least one end configured to be connected to a source of therapeutic gas or fluid. A section of the tube includes a single nasal prong or barrel extending outwardly therefrom. A securing flap or extension is disposed adjacent to, but spaced from, the nasal prong. The securing flap is resilient and extends at an acute angle towards the nasal prong. The securing flap secures the nasal cannula against the side of the user's nostril when the nasal prong is inserted into the nasal opening. The resilience and flexibility of the securing flap insures a comfortable but secure fit, while minimizing any irritation or discomfort on the user. Alternatively, the multi-function nasal cannula may have double-barrels or prongs with corresponding securing flaps.

### CROSS-REFERENCE TO RELATED APPLICATION

This application claims the benefit of U.S. Provisional Patent Application Ser. No. 61/580,617, filed Dec. 27, 2011.

### FIELD OF THE INVENTION

The present invention relates to medical devices, and particularly to a multi-function nasal cannula that provides efficient delivery of therapeutic gases and/or fluids with comfort.

### DESCRIPTION OF THE RELATED ART

Many ailing patients require the use of nasalgastric devices as part of their treatment or recovery. These devices are designed to deliver or administer much needed oxygen, other gases, and/or medication that the patient is physically incapable of performing on their own.

One of the most common types of nasalgastric devices is a double- or dual-barrel or prong nasal cannula. A dual-prong nasal cannula typically includes an elongate tube attached to a source of therapeutic gas, such as oxygen. An adapter is attached to the tube, and the adapter includes pair of nasal prongs providing a pair of outlets for the gas. These prongs are adapted for insertion into the nasal canals of a patient's nose. While the typical nasal cannula performs well, some potential complications can arise from the use thereof. For example, most conventional nasal cannulae are provided in a universal size, having nasal prongs that may not correctly fit in the nasal openings of a patient. Oftentimes, the caregiver must use medical tape to secure the nasal cannula onto the patient's nose. Moreover, the nasalgastric tube can potentially block a nasal opening, hampering the patient's breathing. Extensive use of the typical nasal cannula can also cause dryness inside the nasal passage, which can easily lead to potential blockage of the nasal passage from drying mucous.

Thus, a multi-function nasal cannula solving the aforementioned problems is desired.

### SUMMARY OF THE INVENTION

The multi-function nasal cannula includes an elongate nasalgastric tube having at least one end configured to be connected to a source of therapeutic gas or fluid. A section of the tube includes a single nasal prong or barrel extending outwardly therefrom. A securing flap or extension is disposed adjacent to, but spaced from, the nasal prong. The securing flap is resilient and extends at an acute angle towards the nasal prong. The securing flap secures the nasal cannula against the side of the user's nostril when the nasal prong is inserted into the nasal opening. The resilience and flexibility of the securing flap insures a comfortable but secure fit while minimizing any irritation or discomfort on the user. A double-barrel or prong nasal cannula is also provided with corresponding securing flaps.

These and other features of the present invention will become readily apparent upon further review of the following specification and drawings.

### BRIEF DESCRIPTION OF THE DRAWINGS

FIG. 1 is an environmental, perspective view of a multi-function nasal cannula according to the present invention.

FIG. 2 is a side view of the multi-function nasal cannula shown in FIG. 1.

FIG. 3 is a perspective view of the multi-function nasal cannula shown in FIG. 1.

FIG. 4 is a side view of an alternative embodiment of a multi-function nasal cannula according to the present invention.

Similar reference characters denote corresponding features consistently throughout the attached drawings.

### DETAILED DESCRIPTION OF THE PREFERRED EMBODIMENTS

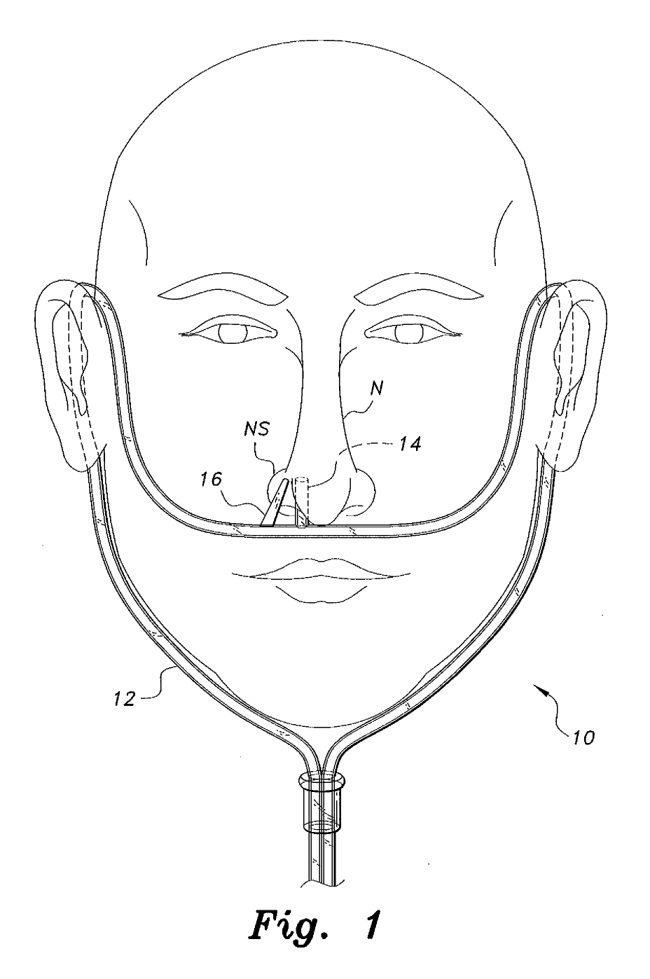

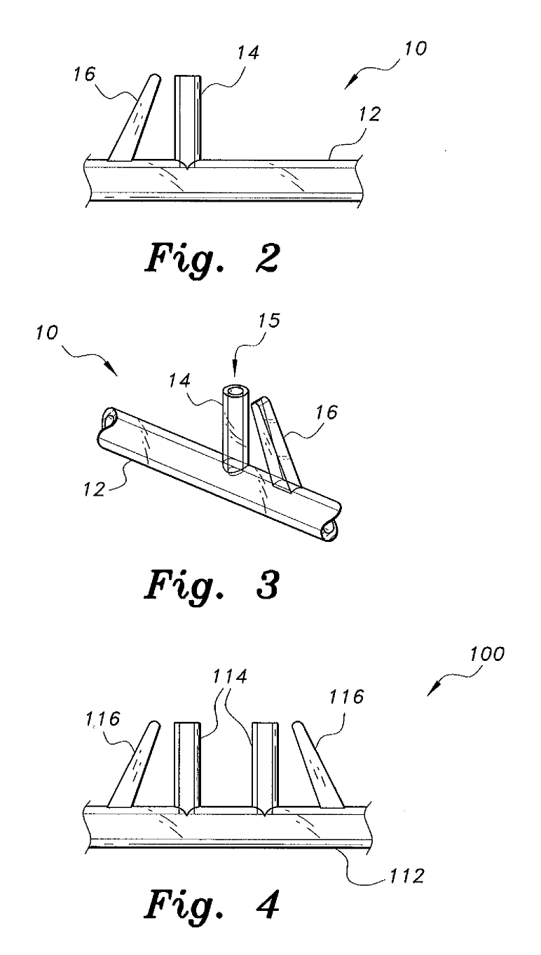

The multi-function nasal cannula, a first embodiment of which is generally referred to in the drawings by the reference number 10, is configured to provide a secure and comfortable fit on the patient's or user's nose and insure a more efficient delivery of therapeutic gas or fluid. As shown in FIGS. 1-3, the multi-function nasal cannula 10 includes an elongate tube 12 having opposite ends, at least one of which is adapted for connection to a source of therapeutic gas (not shown). The length of the tube 12 is preferably long enough so that the multi-function nasal cannula 10 can be connected to the source and easily placed on the patient's head as exemplarily shown in FIG. 1.

A section of the tube 12 includes a single nasal barrel or prong 14 extending outward and substantially orthogonal from the tube 12. The nasal prong 14 is preferably integrally formed on the tube 12. The nasal prong 14 is configured as a short, uniformly cylindrical tube having one end in communication with the hollow interior of the nasalgastric tube 12 and the other, opposite end 15 open to allow the gas or fluid from the tube 12 to escape into a patient's nasal canal. The length of the nasal prong 14 is preferably long enough so that the nasal prong 12 can be inserted into one of the nostrils NS for effective and efficient delivery of the therapeutic gas. The prong 14 defines a lumen that is preferably of uniform diameter from the tube 12 to the open end 15 for maximal gas delivery. The single nasal prong 14 configuration reduces potential blockage of the other or unoccupied nasal opening and minimizes any nasal drying issues. Even if an adjacent portion the nasalgastric tube 12 somehow blocks or hinders air passage, normal breathing will not be further hampered by a nasal prong in the unoccupied nostril NS. If dryness occurs in an occupied nostril NS, the nasal prong 14 can simply be inserted into the other nostril.

To insure that the multi-function nasal cannula 10 is secured on a patient's or user's nose N, the multi-function nasal cannula 10 includes a securing flap or extension 16. The securing flap 16 is disposed adjacent to, but spaced apart from, the nasal prong 14. The spacing provides room for the wall of the nostril NS. The securing flap 16 is preferably an elongate, tapering integral element that extends toward the nasal prong 14 at an acute angle with respect to the nasalgastric tube 12. The nasalgastric tube 12, the nasal prong 14, and the securing flap 16 are all constructed from medical grade elastomeric polymer that exhibits resilience, pliability and suppleness for easy manipulation thereof and durability to withstand sterilization. The flexibility and the angular disposition of the securing flap 16 permits the wall of the nostril NS to be resiliently clamped between the securing flap 16 and nasal prong 14, which helps to prevent the nasal prong 14 from inadvertently sliding out of the nostril NS. Moreover, the tapering configuration of the securing flap 16 provides minimal contact with the nostril NS, which helps minimize skin irritation.

An alternative embodiment of a multi-function nasal cannula 100 is shown in FIG. 4. In this embodiment, the multi-function nasal cannula 100 includes dual nasal prongs or barrels 114 extending orthogonal to a nasalgastric tube 112. A securing flap or extension 116 is disposed adjacent each nasal prong 114. The securing flaps 116 taper and angle inward toward the respective, adjacent nasal prong 114. In this embodiment, the multi-function nasal cannula 100 performs similar to the typical dual barrel nasal cannulae. However, the securing flaps 116 allow the multi-function nasal cannula 100 to be secured on the patient's nose N without tape so that potential blocking of the nasal opening can be substantially reduced.

It is to be understood that the multi-function nasal cannula 10, 100 encompasses a variety of alternatives. For example, the outer edge of the nasal prong 14, 114 at the opening 15 can be rounded to minimize any irritation that may occur from that edge rubbing the inside of the user's nasal passage. Moreover, the nasal prong 14, 114 and the securing flap 16, 116 can be provided in the form of an adapter separately attached to typical nasalgastric tubes. Furthermore, the multi-function nasal cannula 10 can include an additional securing flap 16 of similar configuration to the nasal cannula 100 so that the single nasal prong 14 can be secured by both nostrils NS.

It is to be understood that the present invention is not limited to the embodiments described above, but encompasses any and all embodiments within the scope of the following claims.

I claim:

1. A multi-function nasal cannula, comprising:
an elongate nasalgastric tube having opposite open ends, at least one of the ends being adapted for connection to a source of therapeutic gas;
at least one nasal prong extending outward and orthogonal from a section of the elongate tube, the nasal prong being integral with the nasalgastric tube, the nasal prong being tubular and defining a lumen having one end in communication with the nasalgastric tube and the other end defining an outlet for the therapeutic gas; and
at least one securing flap extending towards the nasal prong, the securing flap being integral with the nasalgastric tube and extending at an acute angle with respect to the nasalgastric tube; the securing flap being adapted for resiliently clamping a wall of a patient's nostril against the nasal prong to prevent the nasal prong being dislodged from the nostril.

2. The multi-function nasal cannula according to claim I, wherein said at least one nasal prong and said at least one securing flap comprise a pair of spaced nasal prongs and a pair of securing flaps, each securing flap being disposed adjacent each nasal prong, both securing flaps extend toward a respective nasal prong and converging in order to clamp both nostrils of the patient.

3. The multi-function nasal cannula according to claim 2, wherein each said securing flap comprises an elongate protrusion integral with said nasalgastric tube, said elongate protrusion tapering towards said respective nasal prong.

4. The multi-function nasal cannula according to claim 1, wherein each said at least one nasal prong comprises an elongate, short tube dimensioned and configured to fit comfortably inside said patient's nostril, the short tube being uniformly cylindrical.

5. The multi-function nasal cannula according to claim 1, wherein said at least one securing flap is resilient in order to resiliently clamp the wall of the patient's nostril against the nasal prong.
